(19) United States
(12) Patent Application Publication
(10) Pub. No.: US 2022/0168170 A1
(43) Pub. Date: Jun. 2, 2022

(54) A DEVICE FOR ALLEVIATING ITCHING

(71) Applicant: Cosi Care LTD, Bexleyheath (GB)

(72) Inventor: Lauren Amy Bell, Bexleyheath (GB)

(21) Appl. No.: 17/434,653

(22) PCT Filed: Feb. 26, 2020
(86) PCT No.: PCT/GB2020/050464

§ 371 Date: Aug. 27, 2021

(30) Foreign Application Priority Data
Feb. 28, 2019 (GB) 1902724.2

(51) Int. Cl. A61H 7/00

(52) U.S. Cl. CPC A61H 7/003; A61H 2201/1692; A61H 2201/0221; A61H 2201/0285; A61H 2201/0214

(57) ABSTRACT

A device (100) for alleviating itching is described. The device (100) includes a body (102) and a plurality of convex protrusions (106) being mounted on the body. In use, the plurality of convex protrusions (106) provides a plurality of contact points on a user's skin.

### FIELD OF THE INVENTION

The present disclosure relates to a device for alleviating itching, in particular for relieving a user's discomfort as a result of irritated skin. The device is particularly suited for relieving a user from itching caused by eczema by providing a replacement for and/or mimicking scratching. The disclosure extends to a corresponding system.

### BACKGROUND TO THE DISCLOSURE

1 in 5 children and 1 in 12 adults suffer with eczema in the UK. 15 million people are currently suffering with the condition and this figure has tripled in the last 30 years. In America this figure is even greater with 1 in 4 children being diagnosed with the condition. The majority of sufferers are diagnosed before the age of 5 and will visit an NHS doctor up to 5 times a year.

Parents raising children with eczema will face a number of challenges, dietary restrictions, clothing limitations, sleep disturbance, maintaining strict moisturising routine but most of all teaching a child not to scratch their skin and make it worse. This is a challenge for a parent as scratching is the child's natural reaction to kill the itching sensation. However, scratching can cause further damage to the skin and may even intensify itching (leading to the “itch-scratch-itch cycle”).

Eczema is a complex autoimmune disease that includes symptoms of painful rashes, cracked dry skin and bleeding. The current treatments are lengthy, clinical and include oral medication and moisturisers that work over a period of time.

### SUMMARY OF THE DISCLOSURE

Aspects and embodiments of the present invention are set out in the appended claims. These and other aspects and embodiments of the invention are also described herein.

In an aspect, there is provided a device for alleviating itching, comprising: a body; a plurality of convex protrusions being mounted on the body; wherein, in use, the plurality of convex protrusions provides a plurality of contact points on a user's skin.

Providing a plurality of convex protrusions which form a plurality of contact points on a user's skin in use mimics and approximates the use of a user's fingers in scratching—however, the convexity of the protrusions reduces skin damage. In particular, the provision of the convex protrusions may allow the device to avoid breaking the skin (i.e. the device is non-invasive) and/or may reduce the possibility of infections.

Preferably, the protrusions are generally hemispherical in shape. Preferably, the surface area of the protrusions is between 0.5 mm² and 630 mm²; more preferably between 4 mm² and 160 mm²; and most preferably between 6 mm² and 100 mm². Preferably, the surface of each of the protrusions is smooth.

Preferably, the protrusions are formed from a heat-conductive material; preferably wherein the material is a metal; more preferably wherein the material is one of: stainless steel; titanium; silver; aluminium; and copper. Preferably, (at least an outer surface of) the body is formed from a heat-resistant material; preferably wherein the material is a polymer material; more preferably wherein the material is silicone.

Preferably, the device further comprises a cooling device for cooling the protrusions, preferably wherein the cooling device is thermally coupled to the protrusions. Preferably, the cooling device is provided internal to the body. Preferably, the cooling device is a Peltier device. Preferably, the cooling device is configured to cool the protrusions to a cooled temperature of between 0 and 20 degrees Celsius; more preferably between 5 and 15 degrees Celsius; yet more preferably between 5 and 10 degrees Celsius. Preferably, the cooling device is configured to cool the protrusions to the cooled temperature within 20 seconds; more preferably within 10 seconds; yet more preferably within 5 seconds; most preferably within 2 seconds. Preferably, the body comprises a vent for the cooling device, the vent being arranged such that, in use, the vent is not covered by any part of a user's body. Optionally, the body comprises a chamber for fitment of an ice pack for cooling the protrusions

Preferably, the device is handheld (i.e. designed to be operated while being held in the hand). Preferably, the body comprises: a first surface on which a formation for alleviating itching is mounted, the formation comprising the plurality of protrusions; and a second surface for supporting a user's palm; wherein, in use, the first surface faces towards a portion of the user's skin to be treated (by use of the device) and the second surface faces away from the user's skin such that the user's palm faces towards the portion of the user's skin to be treated when using the device.

In an aspect, there is provided a device for alleviating itching, comprising: a body, comprising a first surface on which a formation for alleviating itching is mounted; and a second surface for supporting a user's palm; wherein, in use, the first surface faces towards a portion of the user's skin to be treated (by use of the device) and the second surface faces away from the user's skin such that the user's palm faces towards the portion of the user's skin to be treated when using the device. This arrangement may improve the ease of use of the device.

Preferably, the device further comprises a plate on which the formation is provided, the plate being mounted on the first surface of the body, wherein the plate and the formation are unitary. Preferably, the plate is mounted to the body removably, such that the plate can be removed from the body and replaced. Preferably, the plate is substantially concave. Alternatively, the plate is substantially flat. Alternatively, the plate is substantially convex.

Preferably, the body comprises a plurality of recessed portions extending between the first and second surfaces, the recessed portions being configured to receive a user's fingers in use.

Preferably, the first and second surfaces are both shaped generally as a five-pointed star. Preferably, the plurality of protrusions is arranged in a plurality of arrays extending along the points of the star shape of the first surface, wherein each array extends from a centre of the first surface. Preferably, the protrusions vary in size such that the size of each protrusion decreases with increasing distance from the centre of the first surface

Preferably, the body has a child attractive form. For example, the body may be shaped generally as an animal. Preferably, the body is shaped as a starfish.

Preferably, the body comprises an internal battery for powering the device.

Preferably, the internal battery is charged using a USB charging system.

In an aspect, there is provided a system for alleviating itching, comprising: a device as described herein; and a charging dock configured to charge the battery when the device is mounted on the dock. Preferably, the dock comprises a light, wherein the light is configured such that it lights up when the device is charging via the dock.

In an aspect, there is provided a plate comprising a plurality of protrusions configured to couple removably to the device described herein.

In an aspect, there is a provided a plate comprising a plurality of protrusions configured to removably couple to a body of a handheld skincare device wherein the protrusions are configured to interface with a user's skin. Preferably, the plate comprises an attachment mechanism for attaching the plate to the body of the device described herein. Preferably, the attachment mechanism is a surface of the plate.

Furthermore, features implemented in hardware may be implemented in software, and vice versa. Any reference to software and hardware features herein should be construed accordingly.

Any apparatus feature as described herein may also be provided as a method feature, and vice versa. As used herein, means plus function features may be expressed alternatively in terms of their corresponding structure, such as a suitably programmed processor and associated memory.

It should also be appreciated that particular combinations of the various features described and defined in any aspects of the disclosure can be implemented and/or supplied and/or used independently.

The disclosure extends to methods and/or apparatus substantially as herein described with reference to the accompanying drawings. It should be noted that the term “comprising” as used in this document means “consisting at least in part of”. So, when interpreting statements in this document that include the term “comprising”, features other than that or those prefaced by the term may also be present. Related terms such as “comprise” and “comprises” are to be interpreted in the same manner. As used herein, “(s)” following a noun means the plural and/or singular forms of the noun.

As used herein, the term “convex” (when used in relation to an article) preferably connotes an outwardly curving or bulging article (from e.g. a surface); more preferably an article having an outline or surface curved like the exterior of a circle or sphere.

As used herein, the terms “protrusion” and “bulge” are synonymous.

The disclosure will now be described by way of example, with references to the accompanying drawings in which:

### BRIEF DESCRIPTION OF THE DRAWINGS

FIGS. 1A, 1B, and 1C show a device for alleviating itching;

FIG. 2 shows the device and a charging system;

FIGS. 3A, 3B, 3C, 3D, and 3E show plates for use with the device;

FIG. 4 is an electronics schematic diagram of the device; and

FIGS. 5A and 5B show exploded views of the device.

### DETAILED DESCRIPTION

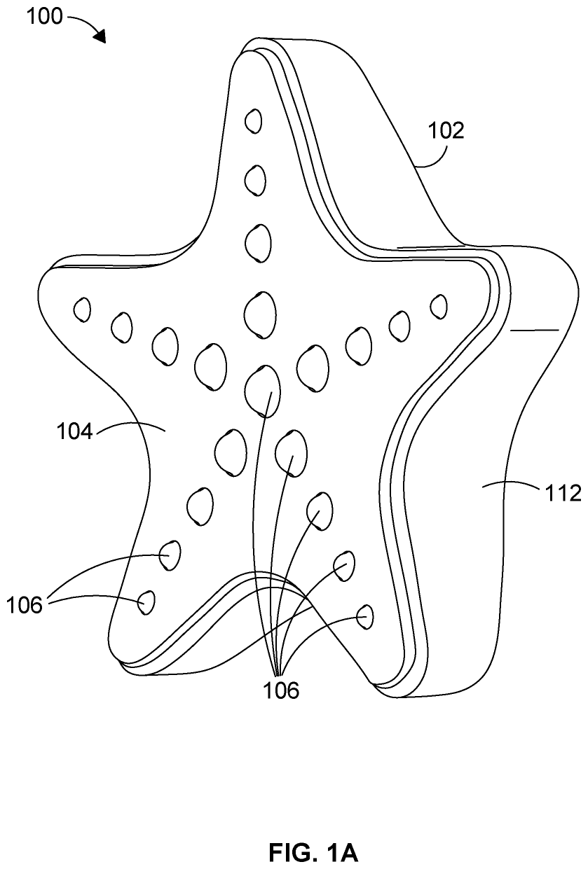

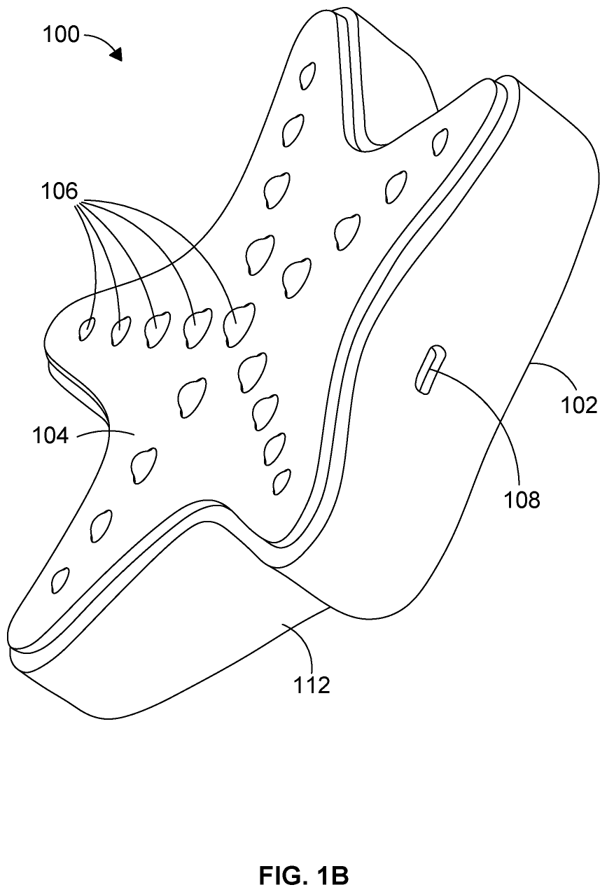

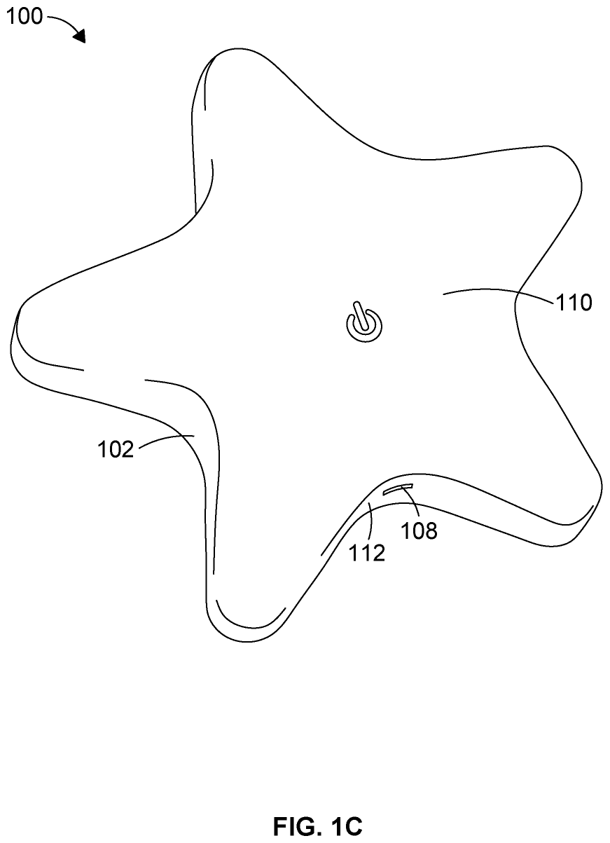

Referring to FIGS. 1A, 1B, and 1C, a handheld skincare device 100 for alleviating itching is shown. The device 100 comprises a body 102, and a plurality of protrusions 106. The protrusions 106 are mounted on (and are part of) a plate 104 (also referred to as a skin interface member), which is in turn mounted on the body 102 (i.e. the protrusions 106 are mounted on the body 102 via the plate 104).

The protrusions 106 are generally convex in shape (i.e. they curve outwardly from the plate 104). In use, this shape of protrusion causes the action of massaging an itching portion of skin using the device 100 to mimic or approximate the sensation of scratching an itching portion of skin so as to alleviate a user's desire to scratch. The shape of the protrusions is configured such that the movement of the protrusions relative to the user's skin provides reduced damage to the skin relative to e.g. a user's fingernails (which are sharp relative to the protrusions and may be unclean). It will be appreciated that the device may thereby provide itch alleviation while reducing (or even eliminating) the prospect of worsening a skin condition by damaging the skin while itching. It will further be appreciated that the problem of worsening a skin condition and/or damaging the skin is particularly acute when the user is a child, since a child's typical low level of impulse control may mean that the user is unable or unwilling to moderate their itching.

The device 100 may provide instant (or near-instant relief) from itching, thereby rendering a user likely to scratch the itching area using their fingernails (or other scratching devices). Avoiding scratching may avoid infection and re-infection, thereby breaking the itch-scratch cycle and allowing the affected skin to heal quicker.

The device 100 is suited to relieve itching resulting from a skin condition such as eczema. Other such conditions causing itching include: cellulitis, chickenpox, erythema multiforme, hand, foot and mouth disease, impetigo, keratosis pilaris (“chicken skin”), measles, molluscum contagiosum, pityriasis rosea, prickly heat, psoriasis, ringworm, scabies, scarlet fever, slapped cheek syndrome, sunburn, burn related injury, and urticaria (hives).

The plate 104 can also be described as the surface from which the protrusions 106 protrude from. The plate 104 comprises a skin interface surface configured to interface with the skin of the user. The skin interface surface of the plate 104 can also be described as comprising a formation for alleviating itching. The plate 104 is generally slightly concave in order to improve conformance to a user's skin (in most use cases, a slightly concave surface will provide improve conformance as compared to a flat surface). Alternatively, the plate 104 is substantially flat. As another alternative, the plate 104 is substantially convex.

The body 102 of the handheld skincare device 100 comprises a first surface on which the plate 104 is mounted. The body 102 of the handheld skincare device 100 further comprises a second surface 110. The second surface 110 can also be described as a surface for supporting a user's palm, or described as an outer or upper surface. Alternatively, the body 102 comprises a member for receiving the plate 104 instead of a first surface. In either embodiment, the plate 104 is configured to be removably mounted or attached to the body 102.

As mentioned, the protrusions 106 are convex, and are preferably substantially hemispherical in shape (i.e. they resemble a portion of, generally half of, a sphere). It will be appreciated that other shapes of protrusion may be used, provided that they are curved and/or smoothed so as to provide a relatively non-abrasive contact with the skin. In alternative embodiments, the protrusions are substantially ovoid, cylindrical, or toroidal. In any embodiment, the protrusions 106 have a substantially curved surface with few to no vertices or edges.

The protrusions 106 have a surface area (i.e. the total area extending above the plate 104) of between 0.5 mm² and 630 mm². More preferably, the surface area is between 4 mm² and 160 mm². Even more preferably the surface area is between 6 mm² and 100 mm². The protrusions 106 are hemispherical, as such, the diameters of the hemispheres corresponding to the surface areas above are approximately between 1 mm and 20 mm, or preferably approximately between 1.5 mm and 10 mm, and most preferably approximately between 2 mm and 8 mm. The protrusions generally vary in size across the plate, such that larger protrusions are towards the centre of the plate, where the size of the protrusions reduces with distance from the centre of the plate. This arrangement of varied sizes of protrusion may allow the user to angle and/or position the device 100 in various different ways so as to provide the most effective relief for their particular requirements.

To reduce chance of reinfection by scratching the user's eczema when using the handheld skincare device 100, the protrusions 106 are made of a smooth or un-textured surface. Preferably the protrusions are made from metal. More preferably, the metal is one of: stainless steel, titanium, silver, aluminium or copper. Alternatively the protrusions may be plated with a metal and preferably any one of the following: stainless steel, titanium, silver, aluminium or copper. The use of metal is preferred as it can have a smooth surface finish, is easily cleaned, and is relatively thermally transmissive.

The plate 104 is shaped generally as a star, in particular a five-pointed star. The body 102 of the device is shaped so as to conform generally to the surface of the plate 104, and includes an upper surface 110 (also referred to as the “second surface”) being generally opposite the first surface (on which the plate 104 and protrusions 106 are provided). The upper surface curves to meet the first surface.

In use, the first surface of the handheld skincare device 100 is positioned towards a portion of a user's skin that requires care/treatment and the second surface faces away from the portion of a user's skin that requires care such that the second surface is facing towards a user's palm. This particular “palm-held” arrangement provides for improved usability of the device (since the device may be placed directly on top of the portion of skin requiring care), in particular for users (such as children) who may struggle to accurately locate the device for use due to their relatively low coordination skills. The device 100 may be sized so as to fit into a child's hand, and its weight may be configured so as to allow a child to lift the device easily with one hand.

The star shape of the first and second surfaces means that recesses 112 are formed between the “points” of the star. These recesses 112 are arranged to receive a user's fingers in use (optionally where several fingers may be used in each recess) so as to provide an easy grip. The star shape of the first and second surfaces may thereby assist in improving the ergonomics of the device while also providing a relatively large area over which the protrusions 106 are provided. The star shape also causes the device to resemble a starfish, which may be a particularly appealing feature for child users.

The body 102 of the device is configured to be generally continuous such that it has no sharp or prominent edges, which might be used for scratching rather than the protrusions 106. This also helps avoid dirt traps. Similarly, the interface between the body 102 and plate 104, and well as the edges of the plate 104 itself, are configured not to be protruding and to be smooth to avoid dirt traps. The device 100 is preferably configured to be waterproof and capable of being cleaned in a dishwasher.

The second surface is made from low allergen material, preferably food grade soft touch silicone. Use of a low allergen material may provide for a better user experience. The silicone may form the bulk of the body 102, or may be overmoulded e.g. on a metal or a plastic frame.

It will be appreciated that eczema sufferers' treatment may involve the regular use of emollients (e.g. moisturisers and steroid creams) on the skin, so the materials selected should not be substantially affected by such emollients. Optionally, the plate 104 or body 102 may comprise a protective coating in this regard.

Eczema can affect all areas of the body. It will be appreciated that the ease of use of the device 100 is such that the device can be used with all areas of the body. Eczema mainly affects wrists, back of the elbows, back of the legs, face, neck, knuckles and fingers. Other affected areas include under the finger nails, lower arms, upper arms, legs, back, chest, lips, ears and scalp.

The device 100 further comprises a cooling device 404, located internal to the body 102. The cooling device 404 is configured to cool the protrusions 106 and/or the plate 104. Cooling the protrusions 106 may improve alleviation of itching by numbing the sensation of the skin and/or by providing a distracting cooling sensation. The cooling device 404 is thermally coupled to the protrusions 106. In an example, the internal side of the plate 104 may physically touch a cool surface of the cooling device 404. The internal side is opposite to the side that touches a user's skin. In an alternative embodiment, thermal paste is used between the cooling device 404 and the protrusions 106 and/or plate 104. The thermal paste acts as a heat transfer element. In another alternative embodiment, heat pipes are used between the protrusions 106 and the cooling device 404. The heat pipes act as a heat transfer element. In another alternative embodiment, the cooling device 404 and protrusions 106 are integral. The protrusions 106 may be solid metal, or alternatively may be generally formed of a flat plate (such that hollow internal cavities corresponding to each of the protrusions are formed).

The cooling device 404 is configured to cool the protrusions 106 to a cooled temperature of between 0 and 20 degrees Celsius. Preferably the temperature of the protrusions when cooled is between 5 and 15 degrees Celsius and even more preferably between 5 and 10 degrees Celsius. A control system is in place to measure the temperature and provide appropriate power to the cooling device 404 to cool the protrusions to the appropriate temperature. In this embodiment, a proportional-integral-derivative (PID) control system is used. Alternative control systems include: proportional, proportional-integral or bang-bang. A person skilled in the art will appreciate that alternative control systems may be used to control temperature. In an alternative embodiment, no control system is used and pre-determined powers and times are used to deliver appropriate output temperatures at the protrusions 106.

The cooling device 404 is configured to cool the protrusions 106 to the target cooled temperature relatively quickly, so as to allow for fast relief of itching. The cooling device 404 is configured to cool the protrusions 106 to the target cooled temperature within 20 seconds. Preferably, the protrusions will reach temperature within 10 seconds. More preferably, the protrusions will reach temperature within 5 seconds. Most preferably, the protrusions will reach temperature within 2 seconds. The use of a cooling device 404 allows the protrusions to be cooled “on the go”, i.e. when the user does not have access to some particular external cooling mechanism. It will be appreciated that the use of a relatively thermally conductive metal plate and a relatively thermally insulating silicone body may allow the device 100 to be used comfortably at the cooled temperature.

The cooling device 404 is generally a Peltier cooler (also referred to as a thermoelectric cooler), which may be used due to its relatively small size and long life.

Alternatively, the cooling device 404 may be a liquid cooling device. A liquid cooling device uses moving water to remove heat from the protrusions 106 and/or plate 104.

In an alternative embodiment, the cooling device 404 is a simple ice pack. The user places the ice pack within the body 102 and the protrusions 106 and/or plate 104 thermally couples with the ice pack. The body 102 comprises an ice pack chamber configured to receive the ice pack.

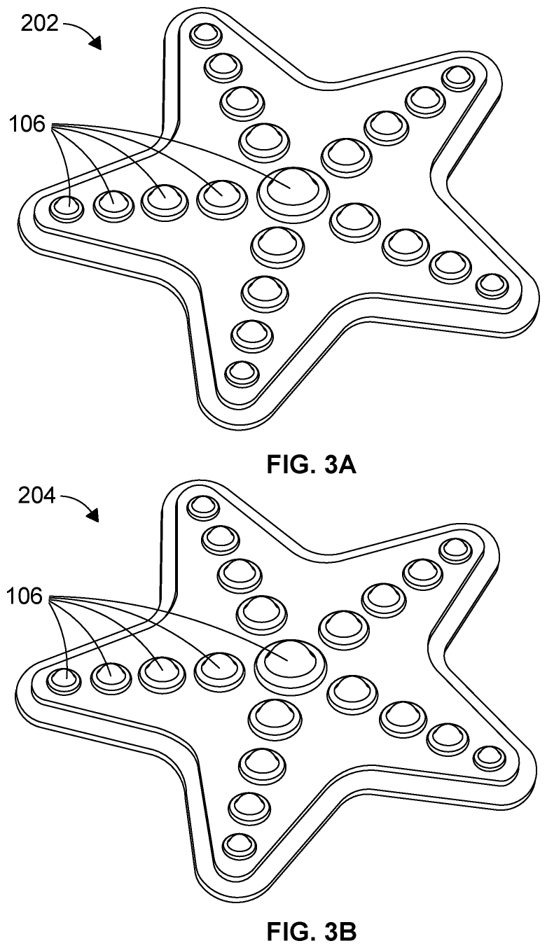

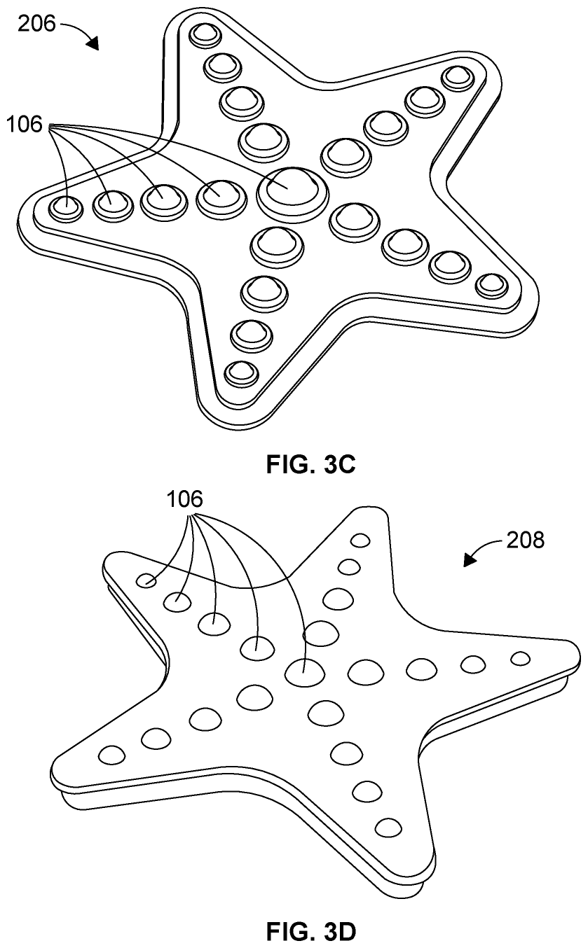

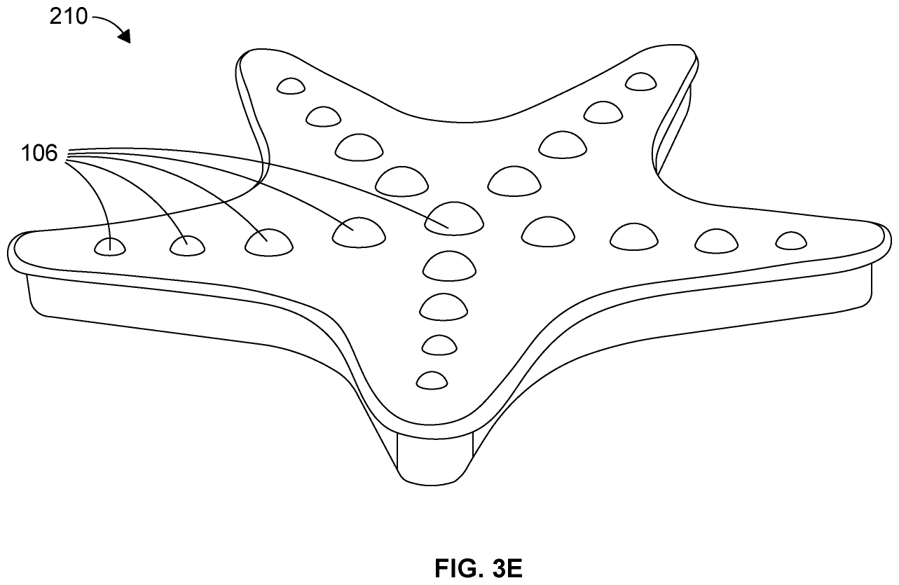

In another alternative embodiment, the protrusions 106 and/or plate 104 are removed from the body 102 and are cooled down. The cooling may be done by placing the protrusions 106 and/or plate 104 into a fridge or a freezer. The thermal mass of the protrusions 106 and/or plate 104 keeps the protrusions 106 cold enough for a user to relieve their itching. Alternatively, the whole device 100 may be placed into a fridge or freezer to cool the protrusions. A person skilled in the art will appreciate that there are other methods of cooling a surface or features of a surface. The protrusions 106 are arranged such that groups of protrusions 106 extend along extended portions of the star shape. The groups extend from the centre of the plate 104. The centre of the plate 104 has a central protrusion where the groups of protrusions 106 meet. The groups of protrusions can also be considered as arrays with a width of one or vectors. In this embodiment, the further away a given protrusion 106 is away from the centre of the plate 104, the smaller the protrusion 106 is. In an alternative embodiment, all of the protrusions 106 are the same size. FIGS. 3A, 3B, 3C, 3D, and 3E show an example pattern where the protrusions 106 get smaller as they extend out from the centre.

When cooling, the cooling device 404 generates excess heat. The heat is expelled from the handheld skincare device 100 away from the protrusions 106. To expel the heat from the handheld skincare device 100, a vent (not shown) is provided in the body 102. The vent is arranged or configured or placed such that in use a user will not block or cover the vent with their finger, palm, hand, or any other part of their body. In particular, the vent may be placed on the end of the extended “points” or portions of the body 102 between the recessed portions, or in a recessed portion. A fan may be used to force any hot air out of the handheld skincare device 100, via “forced convection”. In addition, the fan may be attached to a heat sink and the heat sink is attached to the cooling device 404. Alternatively, a fan may not be required and the hot air escapes the vent without being forced out by a fan, via “natural convection”.

The handheld skincare device 100 further comprises an internal battery for powering the device. In particular, the internal battery is for powering the cooling device 404.

The battery is charged using a charging system. The charging system is a USB or in particular a USB-C charging system similar to a smartphone or other powered handheld device. The charging port 108 provides a point for the user to plug in the power connector. Alternatively, other power transfer ports and/or systems and devices may be used.

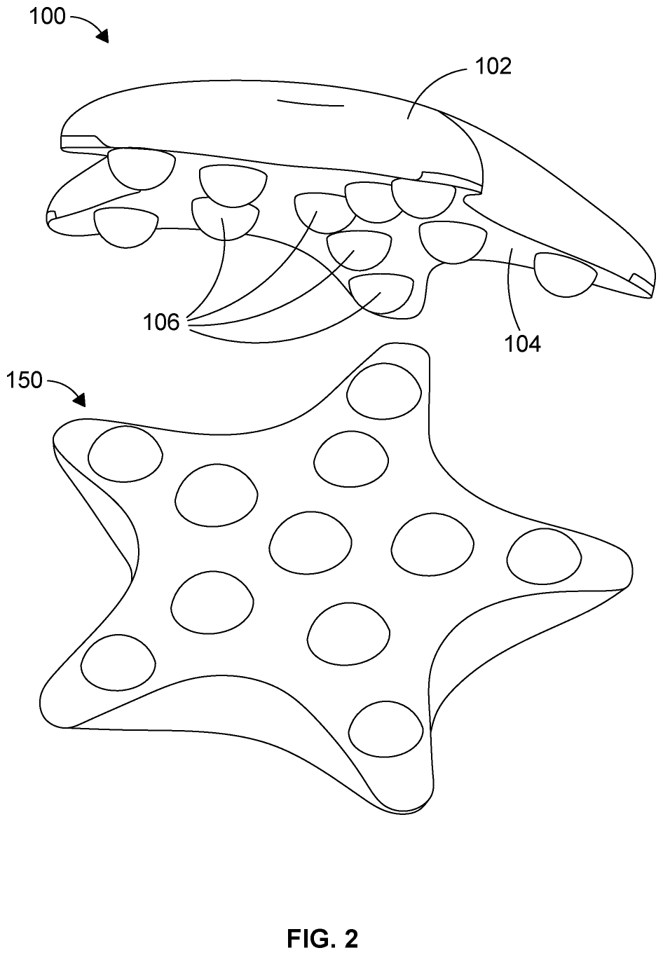

FIG. 2 shows a handheld skincare device 100 with an alternatively shaped body which functions similarly to the previously described body 102 as with reference to FIGS. 1A, 1B, and 1C, including a charging system.

The charging system comprises a charging dock 150. The charging dock 150 is configured to supply power to the handheld skincare device 100. When charging, the charging dock 150 lights up to show that the device is charging, which may act as a nightlight. This may assist child users in locating the device 100 when they wish to use it during the night. The charging dock 150 comprises a surface 152 configured to conform to the plate 104. The surface 152 comprises recesses 154 for receiving the protrusions 106, such that the surface 152 and the plate 104 meet when the device 100 is placed on the dock 150.

When the handheld skincare device 100 is placed on the charging system, metal contacts between the handheld skincare device 100 and the charging system couple and power is delivered to the battery of the handheld skincare device 100. Alternatively, wireless power charging is used instead of metal contacts. In particular, inductive wireless power charging is used in this alternative embodiment. An example of inductive wireless power transfer is the Qi standard.

The device 100 also comprises an on/off switch to enable/disable the cooling device 404. This switch is located on the user surface 110, and may be concealed underneath a soft-touch portion of the silicone material.

The handheld skincare device 100 also comprises a number of LED indicator(s) 414. The LED indicator(s) 414 are configured to show any one or more of the following modes:
when the device is on/off, i.e. the cooling device 404 is enabled,
when the device is cooling,
when the device has reached the appropriate temperature,
when the device is charging,
when the device has finished charging, and/or
when the device has low battery.

The LED indicator(s) 414 indicate the above different modes using any one or more of the following indication techniques:
different coloured LEDs,
LED capable of displaying different colours,
flashing LED(s) at different rates, and/or
flashing alternating colours.

Referring to FIGS. 3A, 3B, 3C, 3D and 3E five alternative embodiments of the plate 104 are shown. The alternative embodiments of FIGS. 3A, 3B, 3C, 3D, and 3E comprise protrusions 106 that decrease in size as they extend from the centre of the plate 104 as with the previous embodiments. A different pattern is shown on FIGS. 3A, 3B, and 3C compared with the embodiment shown in FIGS. 1A, 1B, and 1C.

FIG. 3A shows a plate 202 made from copper.

FIG. 3B shows a plate 204 made from stainless steel.

FIG. 3C shows a plate 206 made from titanium.

FIG. 3D shows a plate 208 made from silver.

FIG. 3E shows a plate 210 made from aluminium.

The plate 104, 202, 204, 206, 208, 210 may be removable from the device 100, and interchangeable. The plates may be sold as a separate unit from the handheld skincare device 100.

Different plates 104, 202, 204, 206, 208, 210 may have different textured surfaces or patterns of protrusions. Having different patterns available provides different options for the user and allows them to choose a pattern and/or texture that suits their needs. In particular, different sizes and/or shapes of protrusions 106 may be used in treatment for alleviating particular conditions.

The plates 104, 202, 204, 206, 208, 210 comprise surfaces or areas that are configured to couple to the body 102 of the handheld skincare device 100. This area or surface can be considered an attachment mechanism. The body 102 of the handheld skincare device 100 also comprises a complementary attachment mechanism to retain or mount the plates 104, 202, 204, 206, 208, 210.

The handheld skincare device 100 may also be used in conjunction with other skin irritation medications. In particular, the plate 104 is made from an inert material such that moisturisers, emollients, creams, or other treatments can be applied to the user interfacing surface of the plate 104.

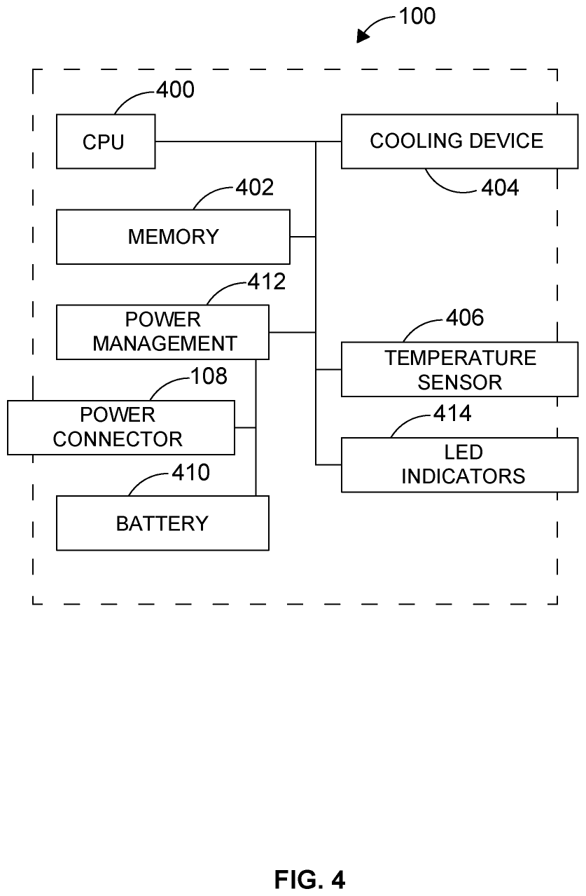

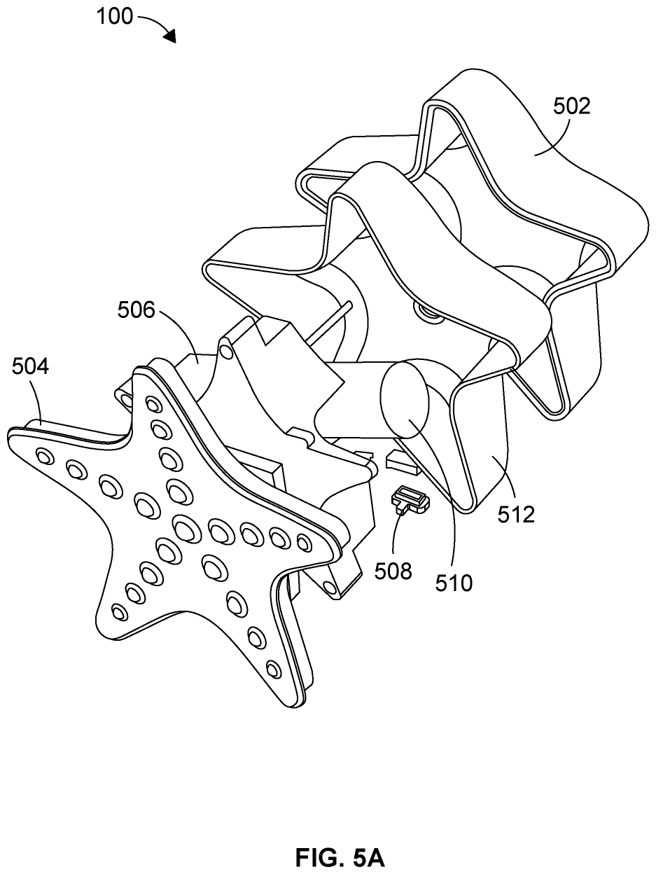

FIG. 4 is a block diagram of the handheld skincare device 100 showing the electronics and computer implemented aspects of the invention including a CPU 400, memory 402, the cooling device 404, temperature sensor 406, power connector 108, battery 410, power management 412, LED indicators 414. The CPU 400 is a computer processor, e.g. a microprocessor. It is arranged to execute instructions in the form of computer executable code, including instructions stored in the memory 402 and/or a storage (not shown). The instructions executed by the CPU 400 include instructions for coordinating operation of the other components of the handheld skincare device 100, such as instructions for controlling the cooling device 404.

The memory 402 is implemented as one or more memory units providing Random Access Memory (RAM) the handheld skincare device 100. In the illustrated embodiment, the memory 402 is a volatile memory, for example in the form of an on-chip RAM integrated with the CPU 400 using System-on-Chip (SoC) architecture. However, in other embodiments, the memory 402 is separate from the CPU 400. The memory 402 is arranged to store the instructions processed by the CPU 400, in the form of computer executable code. Typically, only selected elements of the computer executable code are stored by the memory 402 at any one time, which selected elements define the instructions essential to the operations of the handheld skincare device 100 being carried out at the particular time. In other words, the computer executable code is stored transiently in the memory 402 whilst some particular process is handled by the CPU 400.

The storage (not shown) is provided integrally with the handheld skincare device 100, in the form of a non-volatile memory. The storage is in most embodiments embedded on the same chip as the CPU 400 and the memory 402, using SoC architecture, e.g. by being implemented as a Multiple-Time Programmable (MTP) array. However, in other embodiments, the storage is an embedded or external flash memory, or such like. The storage stores computer executable code defining the instructions processed by the CPU 400. The storage stores the computer executable code permanently or semi-permanently, e.g. until overwritten. That is, the computer executable code is stored in the storage non-transiently. Typically, the computer executable code stored by the storage relates to instructions fundamental to the operation of the CPU 400 and the handheld skincare device 100 more generally.

The cooling device 404 is enabled and disabled according to the temperature of the plate 104 which in this embodiment is measured with the temperature sensor. The control is performed on the CPU 400 in accordance with the control system described previously with reference to FIGS. 1A, 1B, and 1C.

The power system of the handheld skincare device 100 is managed by the power management module 412. The power management module 412 may be implemented on the same chip as the CPU 400 as a System on Chip or as a separate module. The CPU 400 may control aspects of the power management such as enabling low power modes. The CPU 400 is configured to read the current charge state of the battery 410.

The CPU 400 is configured to control the LED indicators 414 as described previously.

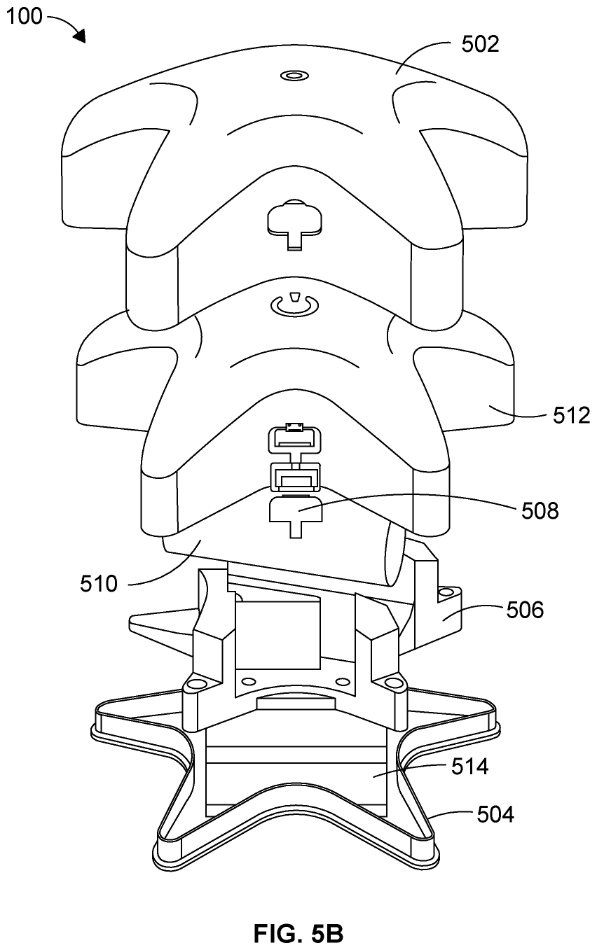

FIGS. 5A and 5B are an exploded view of the device 100 showing many of the physical internal components. The exploded view shows a silicone over-mould 502 (i.e. an outer part of the body), a metal plate with protrusions 504, a solid heat-sink 506, components for the USB-C battery charging system 508, a battery 510, an internal structure 512 (i.e. an inner part of the body), and a cooling device 514. The electronic components and connections are not shown in this exploded view. The silicone over-mould 502 is where the user will grip and hold to device 100. The material could also be a soft touch plastic or high conductive silicone. The metal plate with protrusions 504 is specifically designed for itch relief. As previously described, itch relief may be provided through temperature drop and protrusions working in coordination.

The solid heat-sink 506 is mounted on the plate 504 and surrounds the cooling device 514. The heat-sink 506 is made of metal and provides both a heat sink for the cooling device 514 and some internal structure for the device 100. The heat sink could also be constructed from plastic and be made from a plurality of parts.

The components for the USB-C battery charging system 508 comprise a peel back silicone protection and plug in USB-C lead (lead not shown). The battery 510 (and optionally other electronic components, such as a controller) is provided between the heat-sink 506 and the internal structure 512 may be configured to last for five cycles of 60 seconds before recharging.

The internal structure 512 is a generally shell-like structure which supports the silicone over-mould 502 and acts to enclose the battery 510, solid heat sink 506, and cooling device 514 (where the enclosure is completed by the plate 504). Optionally, the internal structure 512 and silicone over-mould 502 are one single component made from a soft touch plastic instead of two components.

The cooling device 514 is a Peltier device and is mounted on and thermally coupled to the metal plate 504.

The body 102 as described with reference to at least FIG. 1A comprises the silicone over-mould 502 and the internal structure 512.

### Alternatives and Extensions

Although the device 100 has generally been described for alleviating itches resulting from skin conditions such as eczema, it will be appreciated that the device may be used in treating any kind of itching. In particular, the device may be used in alleviating itching sensations resulting from skin disorders such as dandruff or xerosis, scab healing, scar growth, infection (e.g. due to body or head lice), insect bites, allergic reactions, the presence of foreign objects on the skin, photodermatis, any other medical disorder (e.g. diabetes), pregnancy or menopause, and psychiatric disorders.

The device 100 may also be used for alleviating other (non-itching) sensations on the skin, such as pain or paraesthesia, including tingling, pricking, chilling, burning, or numb sensations, formication, and “pins and needles.”

Although the device 100 has generally been described for use with children, it may be equally suitable for use with adults. The device 100 may also be used (in suitably adapted form) for treatment of non-human animals, in particular large mammals such as horses, dogs, cats, pigs, and cows.

The device 100 has been described generally for use in massaging thereby to mimic scratching; however, it may also simply be held onto the area to be treated in order to alleviate itching (in particular when the protrusions 106 are at a cooled temperature).

Optionally, the device 100 further comprises a case configured to protect the plate 104 and protrusions 106 when the device 100 is not in use.

Optionally, the device 100 may be configured to maintain the protrusions 106 at the cooled temperature for a predetermined period of time, such as ten seconds or one minute (i.e. the device may switch off after the predetermined period of time has elapsed). This may assist a user in forming a habit to reduce repetitive itch relief (e.g. by “counting to ten” and then ceasing treatment after that period).

Optionally, the device 100 may not comprise a cooling device and may instead be formed from a single piece of metal, and cooled down by placing the device in the fridge or freezer.

Optionally, the device may hold an arrangement of 4 LEDs for battery power indication.

Optionally, the device may include a charging port which is suitable for a micro USB or C-type USB.

It will be understood that the present disclosure has been described above purely by way of example, and modifications of detail can be made within the scope of the invention.

Reference numerals appearing in the claims are by way of illustration only and shall have no limiting effect on the scope of the claims.

1-14. (canceled)

15. A device for alleviating itching, comprising:
a body, comprising:
a first surface on which a formation for alleviating itching is mounted; and
a second surface for supporting a user's palm;
wherein, in use, the first surface faces towards a portion of the user's skin to be treated and the second surface faces away from the user's skin such that the user's palm faces towards the portion of the user's skin to be treated when using the device,
wherein the device further comprises a plate on which the formation is provided, the plate being mounted on the first surface of the body, wherein the plate and the formation are unitary,
wherein the plate is substantially concave.

16. (canceled)

17. A device according to claim 15, wherein the plate is mounted to the body removably, such that the plate can be removed from the body and replaced.

18. (canceled)

19. A device according to claim 15, wherein the body comprises a plurality of recessed portions extending between the first and second surfaces, the recessed portions being configured to receive a user's fingers in use.

20. A device according to claim 15, wherein the first and second surfaces are both shaped generally as a five-pointed star.

21. A device according to claim 20, wherein the plurality of protrusions is arranged in a plurality of arrays extending along the points of the star shape of the first surface, wherein each array extends from a centre of the first surface.

22. A device according to claim 21, wherein the protrusions vary in size such that the size of each protrusion decreases with increasing distance from the centre of the first surface.

23. A device according to claim 15, wherein the device is handheld (i.e. designed to be operated while being held in the hand).

24. A device according to claim 15, wherein the body comprises an internal battery for powering the device.

25. A system for alleviating itching, comprising:
a device according to claim 24; and
a charging dock configured to charge the battery when the device is mounted on the dock.
